SEQUENCE LISTING (16 sequences)

SEQ ID NO: 1 - DNA, 400 bp, synthetic construct
  source 1..400: mol_type = other DNA; note = nucleotide sequence having the qPV-A01-9089583-A/G
gtttcattttgcagggtatggagggccagacgcgtgcgtggagagtggtggtggtagtgcgcaaggtggccaatggttggccgttggtgggaagcggcggctgacaagatgggagctagggtttgctgctgaaatcttttaagcaaatgggctattagggttttaaaatttgggctcaggtagttttaggattgggctagattggtttagggtattgggctaggttggtttaggcttggtttaggatgagatgggtatgggatttaagtagctttggttattgggtttttgggggtggcccaaaattggcctgtacacctgctaaaagaggtgatataacaagcatagttttagttgacatgggtgtattaaatacaccaaaatattggcattccgcttt

SEQ ID NO: 2 - DNA, 400 bp, synthetic construct
  source 1..400: mol_type = other DNA; note = nucleotide sequence having the qPV-A01-9089583-A/G
gtttcattttgcagggtatggagggccagacgcgtgcgtggagagtggtggtggtagtgcgcaaggtggccaatggttggccgttggtgggaagcggcggctgacaagatgggagctagggtttgctgctgaaatcttttaagcaaatgggctattagggttttaaaatttgggctcaggtagttttaggattgggctaggttggtttagggtattgggctaggttggtttaggcttggtttaggatgagatgggtatgggatttaagtagctttggttattgggtttttgggggtggcccaaaattggcctgtacacctgctaaaagaggtgatataacaagcatagttttagttgacatgggtgtattaaatacaccaaaatattggcattccgcttt

SEQ ID NO: 3 - DNA, 400 bp, synthetic construct
  source 1..400: mol_type = other DNA; note = nucleotide sequence having the qPV-D01-8385035-T/G
aaattttgtaactccaatatatttttaaactttgaatgaaaaagttagtgttgttcagttacaatttctgagtgttcaagattgctttgctgttttattttcttttacttcaatttccattggttaagctttgcaaaatgtatcatgctgctgcctttgttcctacagactaagatgaagattctgttgcaggagatgaatcacatctgttgagtttttgaatcgagttaatccaaaaaagaattgattcaaatgtctactaaagcttcatgcttagactactcattcactttacttcatttaaaaagacatcacttcttaaagtactcaaggaatagaatgaggcaacgcaacctcactaagcactcaaccttcaaaagcttattacgcgccaactacg

SEQ ID NO: 4 - DNA, 400 bp, synthetic construct
  source 1..400: mol_type = other DNA; note = nucleotide sequence having the qPV-D01-8385035-T/G
aaattttgtaactccaatatatttttaaactttgaatgaaaaagttagtgttgttcagttacaatttctgagtgttcaagattgctttgctgttttattttcttttacttcaatttccattggttaagctttgcaaaatgtatcatgctgctgcctttgttcctacagactaagatgaagattctgttgcaggagatgaagcacatctgttgagtttttgaatcgagttaatccaaaaaagaattgattcaaatgtctactaaagcttcatgcttagactactcattcactttacttcatttaaaaagacatcacttcttaaagtactcaaggaatagaatgaggcaacgcaacctcactaagcactcaaccttcaaaagcttattacgcgccaactacg

SEQ ID NO: 5 - DNA, 400 bp, synthetic construct
  source 1..400: mol_type = other DNA; note = nucleotide sequence having the qPV-D05-23825179-G/A
gtagaggagaactattgtttaaaagaagagaaccaaaaggcaaaagttgatcaacaagatggaggtaaaagactcttttaaatgataaacctactagtcttgaggctattcaaaaggatcttatagcaactcaatcgatacttaaaagttcaatacaagtagtgggaaaatgggtgaaacccttacaataggaataagaagccttaagaaaggtggtctaggaatacgttaaaaagaaagaaaaggttatggtcgagagtctaacaatatttgtcaaaaccataaaccgcattgatcgtggagtagtcgagcacattaggctaagatactttaagatgttacataatttgagattgatacaagcagtggtaggaccaatgccaactgctccatgcactac

SEQ ID NO: 6 - DNA, 400 bp, synthetic construct
  source 1..400: mol_type = other DNA; note = nucleotide sequence having the qPV-D05-23825179-G/A
gtagaggagaactattgtttaaaagaagagaaccaaaaggcaaaagttgatcaacaagatggaggtaaaagactcttttaaatgataaacctactagtcttgaggctattcaaaaggatcttatagcaactcaatcgatacttaaaagttcaatacaagtagtgggaaaatgggtgaaacccttacaataggaataagaaaccttaagaaaggtggtctaggaatacgttaaaaagaaagaaaaggttatggtcgagagtctaacaatatttgtcaaaaccataaaccgcattgatcgtggagtagtcgagcacattaggctaagatactttaagatgttacataatttgagattgatacaagcagtggtaggaccaatgccaactgctccatgcactac

SEQ ID NO: 7 - DNA, 400 bp, synthetic construct
  source 1..400: mol_type = other DNA; note = nucleotide sequence having the qPV-D12-42661640-A/T
ttagtaatatgttagaaaataaatggtattcttctcaattcaaaatttttccaaactcgtgttttatatatattatagatttatataattgataagtattttttgtatacatatattaaatatttcttaaaaatatctataaatctaaaacgatatactaaaacaaaccaatactgatacatacaaatttcaatcgaaaaagaatatgtctactggtacattactaactatttttaacccttatcgttactttgctaatcttcctcctcaaatttctcttacattcattacatgttctaacttaaacatcgaaaaacatcttcacaacataaactttgacactatcataaaattatgtatatatattcaaggttcatccaaacgtgtaacccaaactctc

SEQ ID NO: 8 - DNA, 400 bp, synthetic construct
  source 1..400: mol_type = other DNA; note = nucleotide sequence having the qPV-D12-42661640-A/T
ttagtaatatgttagaaaataaatggtattcttctcaattcaaaatttttccaaactcgtgttttatatatattatagatttatataattgataagtattttttgtatacatatattaaatatttcttaaaaatatctataaatctaaaacgatatactaaaacaaaccaatactgatacatacaaatttcaatcgaaaatgaatatgtctactggtacattactaactatttttaacccttatcgttactttgctaatcttcctcctcaaatttctcttacattcattacatgttctaacttaaacatcgaaaaacatcttcacaacataaactttgacactatcataaaattatgtatatatattcaaggttcatccaaacgtgtaacccaaactctc

SEQ ID NO: 9 - DNA, 22 bp, synthetic construct
  source 1..22: mol_type = other DNA; note = upstream primer for amplifying the qPV-A01-9089583-A/G
gtagttttaggattgggctaga

SEQ ID NO: 10 - DNA, 18 bp, synthetic construct
  source 1..18: mol_type = other DNA; note = downstream primer for amplifying the qPV-A01-9089583-A/G
aaagcggaatgccaatat

SEQ ID NO: 11 - DNA, 21 bp, synthetic construct
  source 1..21: mol_type = other DNA; note = upstream primer for amplifying the qPV-D01-8385035-T/G
attctgttgcaggagatgaat

SEQ ID NO: 12 - DNA, 18 bp, synthetic construct
  source 1..18: mol_type = other DNA; note = downstream primer for amplifying the qPV-D01-8385035-T/G
cgtagttggcgcgtaata

SEQ ID NO: 13 - DNA, 24 bp, synthetic construct
  source 1..24: mol_type = other DNA; note = upstream primer for amplifying the qPV-D05-23825179-G/A
aacccttacaataggaataagaag

SEQ ID NO: 14 - DNA, 19 bp, synthetic construct
  source 1..19: mol_type = other DNA; note = downstream primer for amplifying the qPV-D05-23825179-G/A
tagtgcatggagcagttgg

SEQ ID NO: 15 - DNA, 22 bp, synthetic construct
  source 1..22: mol_type = other DNA; note = upstream primer for amplifying the qPV-D12-42661640-A/T
catacaaatttcaatcgaaaaa

SEQ ID NO: 16 - DNA, 20 bp, synthetic construct
  source 1..20: mol_type = other DNA; note = downstream primer for amplifying the qPV-D12-42661640-A/T
gagtttgggttacacgtttg

(19) United States
(12) Patent Application Publication
(10) Pub. No.: US 2025/0320567 A1
(43) Pub. Date: Oct. 16, 2025

(54) QUANTITATIVE TRAIT LOCUS (QTL) AND MOLECULAR MARKER ASSOCIATED WITH HEAT TOLERANCE IN UPLAND COTTON, AND USE THEREOF

(71) Applicant: HUAZHONG AGRICULTURAL UNIVERSITY, Wuhan (CN)

(72) Inventors: Ling MIN, Wuhan (CN); Yizan MA, Wuhan (CN); Longfu ZHU, Wuhan (CN); Xianlong ZHANG, Wuhan (CN)

(21) Appl. No.: 18/859,105

(22) PCT Filed: May 6, 2024
(86) PCT No.: PCT/CN2024/091195

§ 371 Date: Oct. 22, 2024

(30) Foreign Application Priority Data
Apr. 13, 2024 (CN) 202410447186.9

(51) Int. Cl. C12Q 1/6895

(52) U.S. Cl. CPC C12Q 1/6895; C12Q 2600/13; C12Q 2600/156

(57) ABSTRACT

A quantitative trait locus (QTL) and a molecular marker associated with heat tolerance in an upland cotton, and use thereof, belonging to the technical field of biological agriculture. The QTL and the molecular marker thereof associated with a heat tolerance trait of the upland cotton are provided. The QTL is one or more selected from the group consisting of qPV-A01-1, qPV-D01-1, qPV-D05-1, and qPV-D12-1, and is significantly associated with the heat tolerance trait of the upland cotton. In addition, corresponding primers and a detection kit are designed based on the molecular marker closely linked to the QTL, and a method for identifying a heat-tolerant upland cotton is further provided. Therefore, identification and screening of the heat-tolerant upland cotton is achieved, providing a scientific basis for breeding the heat-tolerant upland cotton.

### CROSS REFERENCE TO RELATED APPLICATION

The present application is a national stage application of PCT application No. PCT/CN2024/091195 filed on May 6, 2024, which claims priority to the Chinese Patent Application No. CN202410447186.9, filed with the China National Intellectual Property Administration (CNIPA) on Apr. 13, 2024, and entitled “QUANTITATIVE TRAIT LOCUS (QTL) AND MOLECULAR MARKER ASSOCIATED WITH HEAT TOLERANCE IN UPLAND COTTON, AND USE THEREOF”, both of which are incorporated herein by reference in its entirety.

### REFERENCE TO SEQUENCE LISTING

A computer readable XML file entitled “GWPCTP20240503880_seqlist”, that was created on Jul. 29, 2024, with a file size of about 21,497 bytes, contains the sequence listing for this application, has been filed with this application, and is hereby incorporated by reference in its entirety.

### TECHNICAL FIELD

The present disclosure belongs to the technical field of biological agriculture, and specifically relates to a quantitative trait locus (QTL) and a molecular marker associated with heat tolerance in an upland cotton, and use thereof.

### BACKGROUND

High temperatures can lead to male sterility in cotton, resulting in reduced cotton production and restricting the development of the cotton industry. Breeding heat-tolerant cotton varieties as well as improving the heat tolerance of existing cotton varieties is one of the demands of cotton industry system in China at this stage. However, existing technical research has shown that the heat tolerance phenotype of cotton anthers is a complex agronomic trait regulated by quantitative trait loci (QTLs), with an expression that has cumulative effects. Moreover, due to the instability of heat tolerance phenotype in cotton anther and the non-standardized investigation technology, there are currently no reports on the cloning and identification of heat tolerance QTLs or major effect loci for cotton.

### SUMMARY

An objective of the present disclosure is to provide a QTL and a molecular marker associated with heat tolerance in an upland cotton, and use thereof. The QTL may not only accurately predict and screen a heat-tolerant upland cotton, but also achieve the breeding of an ideal cotton plant type.

To achieve the above objective, the present disclosure provides the following technical solutions:

The present disclosure provides a QTL associated with heat tolerance in an upland cotton, where the QTL is one or more selected from the group consisting of qPV-A01-1, qPV-D01-1, qPV-D05-1, and qPV-D12-1; the qPV-A01-1 is located at 8,809,583 bp to 9,369,583 bp of a chromosome A01 in a genome of the upland cotton; the qPV-D01-1 is located at 8,105,035 and 8,665,035 bp of a chromosome D01 in the genome of the upland cotton; the qPV-D05-1 is located at 23,545,179 bp to 24,105,179 bp of a chromosome D05 in the genome of the upland cotton; the qPV-D12-1 is located at 42,381,640 bp to 42,941,640 bp of a chromosome D12 in the genome of the upland cotton; and the genome of the upland cotton has a version of TM-1_HZAU.v1.1.

The present disclosure further provides a molecular marker tightly linked to the QTL, where the molecular marker is one or more selected from the group consisting of qPV-A01-9089583-A/G, qPV-D01-8385035-T/G, qPV-D05-23825179-G/A, and qPV-D12-42661640-A/T; the qPV-A01-9089583-A/G is located at a base 9,089,583 of the chromosome A01 in the genome of the upland cotton, and the base 9,089,583 is A or G; the qPV-D01-8385035-T/G is located at a base 8,385,035 of the chromosome D01 in the genome of the upland cotton, and the base 8,385,035 is T or G; the qPV-D05-23825179-G/A is located at a base 23,825,179 of the chromosome D05 in the genome of the upland cotton, and the base 23,825,179 is G or A; and the qPV-D12-42661640-A/T is located at a base 42,661,640 of the chromosome D12 in the genome of the upland cotton, and the base 42,661,640 is A or T.

Preferably, the nucleotide sequence having the qPV-A01-9089583-A/G is set forth in SEQ ID NO: 1 or SEQ ID NO: 2; the nucleotide sequence having the qPV-D01-8385035-T/G is set forth in SEQ ID NO: 3 or SEQ ID NO: 4; the nucleotide sequence having the qPV-D05-23825179-G/A is set forth in SEQ ID NO: 5 or SEQ ID NO: 6; and the nucleotide sequence having the qPV-D12-42661640-A/T is set forth in SEQ ID NO: 7 or SEQ ID NO: 8.

The present disclosure further provides a primer set for amplifying the molecular marker, where the primer set is one or more selected from the group consisting of a primer set for amplifying the qPV-A01-9089583-A/G, a primer set for amplifying the qPV-D01-8385035-T/G, a primer set for amplifying the qPV-D05-23825179-G/A, and a primer set for amplifying the qPV-D12-42661640-A/T; an upstream primer for amplifying the qPV-A01-9089583-A/G has the nucleotide sequence set forth in SEQ ID NO: 9 and a downstream primer for amplifying the qPV-A01-9089583-A/G has the nucleotide sequence set forth in SEQ ID NO: 10; an upstream primer for amplifying the qPV-D01-8385035-T/G has the nucleotide sequence set forth in SEQ ID NO: 11 and a downstream primer for amplifying the qPV-D01-8385035-T/G has the nucleotide sequence set forth in SEQ ID NO: 12; an upstream primer for amplifying the qPV-D05-23825179-G/A has the nucleotide sequence set forth in SEQ ID NO: 13 and a downstream primer for amplifying the qPV-D05-23825179-G/A has the nucleotide sequence set forth in SEQ ID NO: 14; and an upstream primer for amplifying the qPV-D12-42661640-A/T has the nucleotide sequence set forth in SEQ ID NO: 15 and a downstream primer for amplifying the qPV-D12-42661640-A/T has the nucleotide sequence set forth in SEQ ID NO: 16.

The present disclosure further provides a detection kit, including the primer set.

The present disclosure further provides use of the QTL, the molecular marker, the primer set, or the detection kit in one or more of identification, screening, and breeding of an upland cotton tolerant to a high temperature.

In some embodiments, the high temperature is greater than or equal to 35° C.

The present disclosure further provides a method for identifying a heat-tolerant upland cotton, including the following steps:
subjecting a genomic DNA of an upland cotton to be tested to PCR amplification using the primer set to obtain a PCR amplification product; detecting and analyzing a genotype of the PCR amplification product; if the molecular marker qPV-A01-9089583-A/G is a heat-tolerant homozygous genotype AA, the molecular marker qPV-D01-8385035-T/G is a heat-tolerant homozygous genotype TT, the molecular marker qPV-D05-23825179-G/A is a heat-tolerant homozygous genotype GG, and the molecular marker qPV-D12-42661640-A/T is a heat-tolerant homozygous genotype AA in the PCR amplification product, determining that the upland cotton to be tested is the heat-tolerant upland cotton; and if at least one of the molecular markers qPV-A01-9089583-A/G, qPV-D01-8385035-T/G, qPV-D05-23825179-G/A, and qPV-D12-42661640-A/T in the PCR amplification product is a non-heat-tolerant homozygous genotype, determining that the upland cotton to be tested is a non-heat-tolerant upland cotton.

In some embodiments, a reaction system of the PCR amplification has a volume of 20 μL and includes: 2 μL of a 10× Buffer, 1 μL of a 75 ng/μL to 100 ng/μL genomic DNA template, 0.5 μL of a 10 μM upstream primer, 0.5 μL of a 10 μM downstream primer, 0.3 μL of a dNTP mix, 0.2 μL of a Taq enzyme, and supplementing to 20 μL with ddH₂O.

In some embodiments, a reaction procedure of the PCR amplification includes: initial denaturation at 95° C. for 5 min; 35 cycles of denaturation at 95° C. for 30 s, annealing at 53° C. to 62° C. for 30 s, and extension at 72° C. for 30 s; and extension at 72° C. for 30 s.

### Beneficial Effects

The QTL and the molecular marker thereof associated with a heat tolerance trait of the upland cotton are provided. The QTL is one or more selected from the group consisting of qPV-A01-1, qPV-D01-1, qPV-D05-1, and qPV-D12-1, and is significantly associated with the heat tolerance trait of the upland cotton. In addition, corresponding primers and a detection kit are designed based on the molecular marker closely linked to the QTL. Therefore, prediction and screening of the heat-tolerant upland cotton is achieved, providing a scientific basis for breeding the heat-tolerant upland cotton.

Based on the above technical advantages, the present disclosure further provides a method for identifying a heat-tolerant upland cotton, including the following steps: subjecting a genomic DNA of an upland cotton to be tested to PCR amplification using the primer set to obtain a PCR amplification product; detecting and analyzing a genotype of the PCR amplification product; the molecular marker qPV-A01-9089583-A/G is a homozygous genotype AA, the molecular marker qPV-D01-8385035-T/G is a homozygous genotype TT, the molecular marker qPV-D05-23825179-G/A is a homozygous genotype GG, and the molecular marker qPV-D12-42661640-A/T is a homozygous genotype AA in the PCR amplification fragment, determining that the upland cotton to be tested is the heat-tolerant upland cotton. Experiments have shown that the technical solutions provided by the present disclosure may accurately identify and screen the heat tolerance trait of upland cotton and achieve the breeding of an ideal cotton plant type.

### BRIEF DESCRIPTION OF THE DRAWINGS

To illustrate the examples of the present disclosure or the technical solutions in the prior art more clearly, the accompanying drawings required in the examples will be briefly introduced below.

FIGS. 1A-IF show the identification of QTL related to heat tolerance in upland cotton by genome-wide association study (GWAS) in Example 1;

FIGS. 2A-2D show the genotype effect analysis of the heat tolerance QTL in Example 2;

FIGS. 3A-3C show the utilization of the heat tolerance QTL in the natural population of upland cotton in Example 2;

FIG. 4 shows the molecular marker development of SNP variations within four QTLs in Example 3;

FIGS. 5A-5B show the feasibility verification of the four heat-tolerant molecular markers in Example 4 in commercial hybrids;

FIGS. 6A-6B show the results of molecular marker-assisted upland cotton routine breeding in Example 4;

FIGS. 7A-7B show the results of molecular marker-assisted upland cotton hybrid breeding in Example 4.

### DETAILED DESCRIPTION OF THE EMBODIMENTS

The present disclosure provides a QTL associated with heat tolerance in an upland cotton, where the QTL is one or more selected from the group consisting of qPV-A01-1, qPV-D01-1, qPV-D05-1, and qPV-D12-1, preferably the qPV-A01-1, the qPV-D01-1, the qPV-D05-1, and the qPV-D12-1; the qPV-A01-1 is located at 8,809,583 bp to 9,369,583 bp of a chromosome A01 in a genome of the upland cotton; the qPV-D01-1 is located at 8,105,035 and 8,665,035 bp of a chromosome D01 in the genome of the upland cotton; the qPV-D05-1 is located at 23,545,179 bp to 24,105,179 bp of a chromosome D05 in the genome of the upland cotton; the qPV-D12-1 is located at 42,381,640 bp to 42,941,640 bp of a chromosome D12 in the genome of the upland cotton; and the genome of the upland cotton has a version of TM-1_HZAU.v1.1. The QTL is significantly associated with the heat tolerance trait of upland cotton.

The present disclosure further provides a molecular marker tightly linked to the QTL, where the molecular marker is one or more selected from the group consisting of qPV-A01-9089583-A/G, qPV-D01-8385035-T/G, qPV-D05-23825179-G/A, and qPV-D12-42661640-A/T, preferably the qPV-A01-9089583-A/G, the qPV-D01-8385035-T/G, the qPV-D05-23825179-G/A, and the qPV-D12-42661640-A/T.

In the present disclosure, the qPV-A01-9089583-A/G is located at a base 9,089,583 of the chromosome A01 in the genome of the upland cotton, and the base 9,089,583 is A or G; the nucleotide sequence having the qPV-A01-9089583-A/G is preferably set forth in SEQ ID NO: 1 or SEQ ID NO: 2; the nucleotide sequence set forth in SEQ ID NO: 1 is specifically: 5′-GTTTCATTTTGCAGGGTATGGAGGGCCAGACGCGTGCGTGGAGAGTGGTGGTGGTAG TGCGCAAGGTGGCCAATGGTTGGCCGTTGGTGGGAAGCGGCGGCTGACAAGATGGGA GCTAGGGTTTGCTGCTGAAATCTTTTAAGCAAATGGGCTATTAGGGTTTTAAAATTTGG GCTCAGGTAGTTTTAGGATTGGGCTAGATTGGTTTAGGGTATTGGGCTAGGTTGGTTTA GGCTTGGTTTAGGATGAGATGGGTATGGGATTTAAGTAGCTTTGGTTATTGGGTTTTTG GGGGTGGCCCAAAATTGGCCTGTACACCTGCTAAAAGAGGTGATATAACAAGCATAGT TTTAGTTGACATGGGTGTATTAAATACACCAAAATATTGGCATTCCGCTTT-3′; the nucleotide sequence set forth in SEQ ID NO: 2 is specifically: 5′-GTTTCATTTTGCAGGGTATGGAGGGCCAGACGCGTGCGTGGAGAGTGGTGGTGGTAG TGCGCAAGGTGGCCAATGGTTGGCCGTTGGTGGGAAGCGGCGGCTGACAAGATGGGA GCTAGGGTTTGCTGCTGAAATCTTTTAAGCAAATGGGCTATTAGGGTTTTAAAATTTGG GCTCAGGTAGTTTTAGGATTGGGCTAGGTTGGTTTAGGGTATTGGGCTAGGTTGGTTTA GGCTTGGTTTAGGATGAGATGGGTATGGGATTTAAGTAGCTTTGGTTATTGGGTTTTTG GGGGTGGCCCAAAATTGGCCTGTACACCTGCTAAAAGAGGTGATATAACAAGCATAGT TTTAGTTGACATGGGTGTATTAAATACACCAAAATATTGGCATTCCGCTTT-3′.

In the present disclosure, the qPV-D01-8385035-T/G is located at a base 8,385,035 of the chromosome D01 in the genome of the upland cotton, and the base 8,385,035 is T or G; the nucleotide sequence having the qPV-A01-8385035-T/G is preferably set forth in SEQ ID NO: 3 or SEQ ID NO: 4; the nucleotide sequence set forth in SEQ ID NO: 3 is specifically: 5′-AAATTTTGTAACTCCAATATATTTTTAAACTTTGAATGAAAAAGTTAGTGTTGTTCAG TTACAATTTCTGAGTGTTCAAGATTGCTTTGCTGTTTTATTTTCTTTTACTTCAATTTCCA TTGGTTAAGCTTTGCAAAATGTATCATGCTGCTGCCTTTGTTCCTACAGACTAAGATGA AGATTCTGTTGCAGGAGATGAATCACATCTGTTGAGTTTTTGAATCGAGTTAATCCAAA AAAGAATTGATTCAAATGTCTACTAAAGCTTCATGCTTAGACTACTCATTCACTTTACT TCATTTAAAAAGACATCACTTCTTAAAGTACTCAAGGAATAGAATGAGGCAACGCAAC CTCACTAAGCACTCAACCTTCAAAAGCTTATTACGCGCCAACTACG-3′; the nucleotide sequence set forth in SEQ ID NO: 4 is specifically: 5′-AAATTTTGTAACTCCAATATATTTTTAAACTTTGAATGAAAAAGTTAGTGTTGTTCAG TTACAATTTCTGAGTGTTCAAGATTGCTTTGCTGTTTTATTTTCTTTTACTTCAATTTCCA TTGGTTAAGCTTTGCAAAATGTATCATGCTGCTGCCTTTGTTCCTACAGACTAAGATGA AGATTCTGTTGCAGGAGATGAAGCACATCTGTTGAGTTTTTGAATCGAGTTAATCCAAA AAAGAATTGATTCAAATGTCTACTAAAGCTTCATGCTTAGACTACTCATTCACTTTACT TCATTTAAAAAGACATCACTTCTTAAAGTACTCAAGGAATAGAATGAGGCAACGCAAC CTCACTAAGCACTCAACCTTCAAAAGCTTATTACGCGCCAACTACG-3′.

In the present disclosure, the qPV-D05-23825179-G/A is located at a base 23,825,179 of the chromosome D05 in the genome of the upland cotton, and the base 23,825,179 is G or A; the nucleotide sequence having the qPV-D05-23825179-G/A is preferably set forth in SEQ ID NO: 5 or SEQ ID NO: 6; the nucleotide sequence set forth in SEQ ID NO: 5 is specifically: 5′-GTAGAGGAGAACTATTGTTTAAAAGAAGAGAACCAAAAGGCAAAAGTTGATCAACA AGATGGAGGTAAAAGACTCTTTTAAATGATAAACCTACTAGTCTTGAGGCTATTCAAA AGGATCTTATAGCAACTCAATCGATACTTAAAAGTTCAATACAAGTAGTGGGAAAATG GGTGAAACCCTTACAATAGGAATAAGAAGCCTTAAGAAAGGTGGTCTAGGAATACGTT AAAAAGAAAGAAAAGGTTATGGTCGAGAGTCTAACAATATTTGTCAAAACCATAAACC GCATTGATCGTGGAGTAGTCGAGCACATTAGGCTAAGATACTTTAAGATGTTACATAA TTTGAGATTGATACAAGCAGTGGTAGGACCAATGCCAACTGCTCCATGCACTAC-3′; the nucleotide set forth in SEQ ID NO: 6 is sequence specifically: 5′-GTAGAGGAGAACTATTGTTTAAAAGAAGAGAACCAAAAGGCAAAAGTTGATCAACA AGATGGAGGTAAAAGACTCTTTTAAATGATAAACCTACTAGTCTTGAGGCTATTCAAA AGGATCTTATAGCAACTCAATCGATACTTAAAAGTTCAATACAAGTAGTGGGAAAATG GGTGAAACCCTTACAATAGGAATAAGAAACCTTAAGAAAGGTGGTCTAGGAATACGTT AAAAAGAAAGAAAAGGTTATGGTCGAGAGTCTAACAATATTTGTCAAAACCATAAACC GCATTGATCGTGGAGTAGTCGAGCACATTAGGCTAAGATACTTTAAGATGTTACATAA TTTGAGATTGATACAAGCAGTGGTAGGACCAATGCCAACTGCTCCATGCACTAC-3′.

In the present disclosure, the qPV-D12-42661640-A/T is located at a base 42,661,640 of the chromosome D12 in the genome of the upland cotton, and the base 42,661,640 is A or T; the nucleotide sequence having the qPV-D12-42661640-A/T is preferably set forth in SEQ ID NO: 7 or SEQ ID NO: 8; the nucleotide sequence set forth in SEQ ID NO: 7 is specifically: 5′-TTAGTAATATGTTAGAAAATAAATGGTATTCTTCTCAATTCAAAATTTTTCCAAACTC GTGTTTTATATATATTATAGATTTATATAATTGATAAGTATTTTTTGTATACATATATTA AATATTTCTTAAAAATATCTATAAATCTAAAACGATATACTAAAACAAACCAATACTG ATACATACAAATTTCAATCGAAAAAGAATATGTCTACTGGTACATTACTAACTATTTTT AACCCTTATCGTTACTTTGCTAATCTTCCTCCTCAAATTTCTCTTACATTCATTACATGTT CTAACTTAAACATCGAAAAACATCTTCACAACATAAACTTTGACACTATCATAAAATTA TGTATATATATTCAAGGTTCATCCAAACGTGTAACCCAAACTCTC-3′; the nucleotide sequence set forth in SEQ ID NO: 8 is specifically: 5′-TTAGTAATATGTTAGAAAATAAATGGTATTCTTCTCAATTCAAAATTTTTCCAAACTC GTGTTTTATATATATTATAGATTTATATAATTGATAAGTATTTTTTGTATACATATATTA AATATTTCTTAAAAATATCTATAAATCTAAAACGATATACTAAAACAAACCAATACTG ATACATACAAATTTCAATCGAAAATGAATATGTCTACTGGTACATTACTAACTATTTTT AACCCTTATCGTTACTTTGCTAATCTTCCTCCTCAAATTTCTCTTACATTCATTACATGTT CTAACTTAAACATCGAAAAACATCTTCACAACATAAACTTTGACACTATCATAAAATTA TGTATATATATTCAAGGTTCATCCAAACGTGTAACCCAAACTCTC-3′. The molecular markers qPV-A01-9089583-A/G, qPV-D01-8385035-T/G, qPV-D05-23825179-G/A, and qPV-D12-42661640-A/T may specifically mark the upland cotton with heat tolerance trait.

The present disclosure further provides a primer set for amplifying the molecular marker, where the primer set is one or more selected from the group consisting of a primer set for amplifying the qPV-A01-9089583-A/G, a primer set for amplifying the qPV-D01-8385035-T/G, a primer set for amplifying the qPV-D05-23825179-G/A, and a primer set for amplifying the qPV-D12-42661640-A/T, preferably the primer set for amplifying the qPV-A01-9089583-A/G, the primer set for amplifying the qPV-D01-8385035-T/G, the primer set for amplifying the qPV-D05-23825179-G/A, and the primer set for amplifying the qPV-D12-42661640-A/T.

In the present disclosure, the nucleotide sequence of the upstream primer for amplifying the qPV-A01-9089583-A/G is set forth in SEQ ID NO: 9, specifically 5′-GTAGTTTTAGGATTGGGCTAGA-3′; the nucleotide sequence of the downstream primer for amplifying the qPV-A01-9089583-A/G is set forth in SEQ ID NO: 10, specifically 5′-AAAGCGGAATGCCAATAT-3′. the nucleotide sequence of the upstream primer for amplifying the qPV-D01-8385035-T/G is set forth in SEQ ID NO: 11, specifically 5′-ATTCTGTTGCAGGAGATGAAT-3′; the nucleotide sequence of the downstream primer for amplifying the qPV-D01-8385035-T/G is set forth in SEQ ID NO: 12, specifically 5′-CGTAGTTGGCGCGTAATA-3′. the nucleotide sequence of the upstream primer for amplifying the qPV-D05-23825179-G/A is set forth in SEQ ID NO: 13, specifically 5′-AACCCTTACAATAGGAATAAGAAG-3′; the nucleotide sequence of the downstream primer for amplifying the qPV-D05-23825179-G/A is set forth in SEQ ID NO: 14, specifically 5′-TAGTGCATGGAGCAGTTGG-3′. the nucleotide sequence of the upstream primer for amplifying the qPV-D12-42661640-A/T is set forth in SEQ ID NO: 15, specifically 5′-CATACAAATTTCAATCGAAAAA-3′; the nucleotide sequence of the downstream primer for amplifying the qPV-D12-42661640-A/T is set forth in SEQ ID NO: 16, specifically 5′-GAGTTTGGGTTACACGTTTG-3′. The primer set may specifically detect the molecular marker closely linked to QTL, thereby realizing the prediction and screening of heat-tolerant upland cotton, and providing a scientific basis for breeding different heat-tolerant upland cotton varieties.

The present disclosure further provides a detection kit, including the primer set. In the present disclosure, the detection kit further preferably includes a PCR amplification reagent; the PCR amplification reagent preferably includes a DNA polymerase, dNTPs, and Mg²⁺; there are no special limitations on source and amount of the DNA polymerase, dNTPs, and Mg²⁺, and commercially available products in the field may be used.

The present disclosure further provides use of the QTL, the molecular marker, the primer set, or the detection kit in one or more of identification, screening, and breeding of an upland cotton tolerant to a high temperature; where the high temperature is preferably greater than or equal to 35° C., more preferably greater than or equal to 35° C. for 3 consecutive days.

The present disclosure further provides a method for identifying a heat-tolerant upland cotton, including the following steps: subjecting a genomic DNA of an upland cotton to be tested to PCR amplification using the primer set to obtain a PCR amplification product; detecting and analyzing a genotype of the PCR amplification product; when the molecular marker qPV-A01-9089583-A/G is a homozygous genotype AA, the molecular marker qPV-D01-8385035-T/G is a homozygous genotype TT, the molecular marker qPV-D05-23825179-G/A is a homozygous genotype GG, and the molecular marker qPV-D12-42661640-A/T is a homozygous genotype AA in the PCR amplification product, determining that the upland cotton to be tested is the heat-tolerant upland cotton; and when at least one of the molecular markers qPV-A01-9089583-A/G, qPV-D01-8385035-T/G, qPV-D05-23825179-G/A, and qPV-D12-42661640-A/T is a non-homozygous genotype in the PCR amplification product, determining that the upland cotton to be tested is a non-heat-tolerant upland cotton.

In the present disclosure, a genomic DNA of the upland cotton to be tested is preferably extracted to obtain a template DNA. There are no special requirements for extraction method and source of the template DNA, and techniques well known in the art may be used.

In the present disclosure, the template DNA is subjected to PCR amplification using the primer set to obtain the PCR amplification product. In the present disclosure, a reaction system of the PCR amplification has a volume of 20 μL and includes preferably: 2 μL of a 10× Buffer, 1 μL of a 75 ng/μL to 100 ng/μL genomic DNA template, 0.5 μL of a 10 UM upstream primer, 0.5 μL of a 10 μM downstream primer, 0.3 L of a dNTP mix, 0.2 μL of a Taq enzyme, and supplementing to 20 μL with ddH₂O. Preferably, a reaction procedure of the PCR amplification includes: initial denaturation at 95° C. for 5 min; 35 cycles of denaturation at 95° C. for 30 s, annealing at 53° C. to 62° C. for 30 s, and extension at 72° C. for 30 s; and extension at 72° C. for 30 s. In the present disclosure, the annealing temperature is further preferably different according to different templates, specifically: the qPV-A01-9089583-A/G is amplified at an annealing temperature of most preferably 62° C.; the qPV-D01-8385035-T/G is amplified at an annealing temperature of most preferably 61° C.; the qPV-D05-23825179-G/A is amplified at an annealing temperature of most preferably 61° C.; and the qPV-D12-42661640-A/T is amplified at an annealing temperature of most preferably 55° C.

In the present disclosure, a genotype of the PCR amplification product is detected and analyzed. There are no special requirements on method and reagents for detecting the genotype of the PCR amplification product, and techniques well known in the art may be used. When the molecular marker qPV-A01-9089583-A/G is a homozygous genotype AA, the molecular marker qPV-D01-8385035-T/G is a homozygous genotype TT, the molecular marker qPV-D05-23825179-G/A is genotype GG, and the molecular marker qPV-D12-42661640-A/T is a homozygous genotype AA in the PCR amplification product, determining that the upland cotton to be tested is the heat-tolerant upland cotton; and when at least one of the molecular markers qPV-A01-9089583-A/G, qPV-D01-8385035-T/G, qPV-D05-23825179-G/A, and qPV-D12-42661640-A/T is a non-homozygous genotype in the PCR amplification product, determining that the upland cotton to be tested is a non-heat-tolerant upland cotton.

Experiments have shown that the technical solutions provided by the present disclosure may accurately identify and screen the heat tolerance trait of upland cotton and then achieve the breeding of an ideal cotton plant type.

In order to further illustrate the present disclosure, the QTL and the molecular marker associated with heat tolerance in an upland cotton, and the use thereof provided by the present disclosure are described in detail below with reference to the accompanying drawings and examples, but the accompanying drawings and the examples should not be construed as limiting the protection scope of the present disclosure.

### Example 1

### Identification of QTL Related to Heat Tolerance in Upland Cotton by GWAS

The resequencing data of 376 upland cotton germplasms in a publicly published article (article DOI: 10.1038/s41588-021-00844-9) were used to construct an SNP natural variation map of upland cotton (the variation map information had been made public at DOI: 10.1038/s41588-023-01530-8, and the genome version of upland cotton was TM-1_HZAU.v1.1, which could be obtained at cottongen.org/organism/1033). Combined with a rapid pollen activity quantification system established in the laboratory in an early stage (the pollen activity quantification system had been published in ZL201910010240.2), the pollen heat tolerance phenotype of the above 376 upland cotton natural populations was obtained, and GWAS was conducted as follows:
1. Population genetics analysis of 376 upland cotton varieties was conducted based on natural variation maps, including population genetic structure, principal component analysis, linkage disequilibrium, and species occurrence tree construction.
2. The anther heat tolerance phenotype of 376 upland cotton varieties was investigated in Turpan, Xinjiang, Aksu, Xinjiang, Korla, Xinjiang, Wuhan, Hubei, and Ezhou, Hubei in 2016, 2018, 2019, 2021, 2022, and 2023. GWAS was conducted using a mixed linear model called by EMMAX software, with the following equation: y=Xb+Zu+e.

y represented the observation value vector; b represented the fixed factor effect value vector; X represented the fixed factor relationship matrix; u represented the random factor effect value vector; Z represented the random factor relationship matrix; e represented the residual vector; and

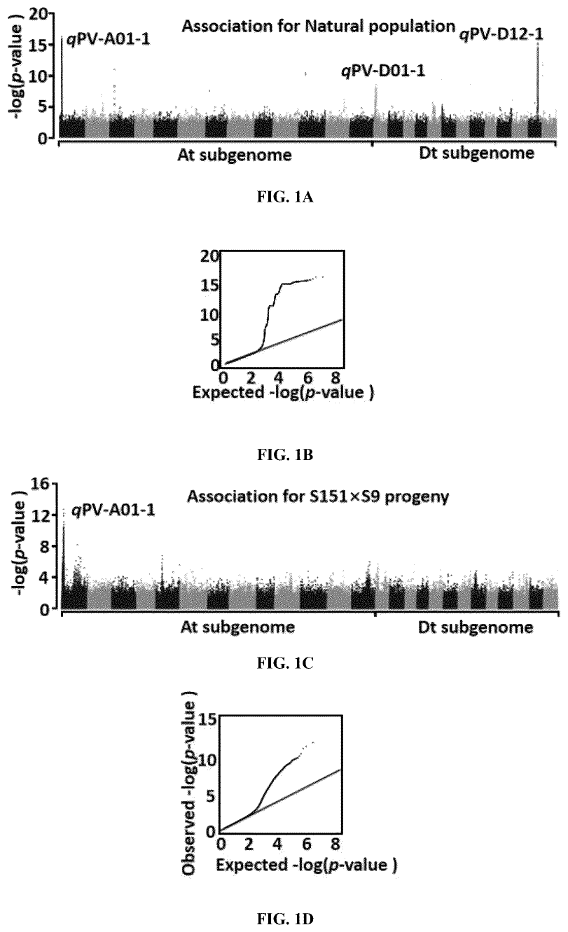

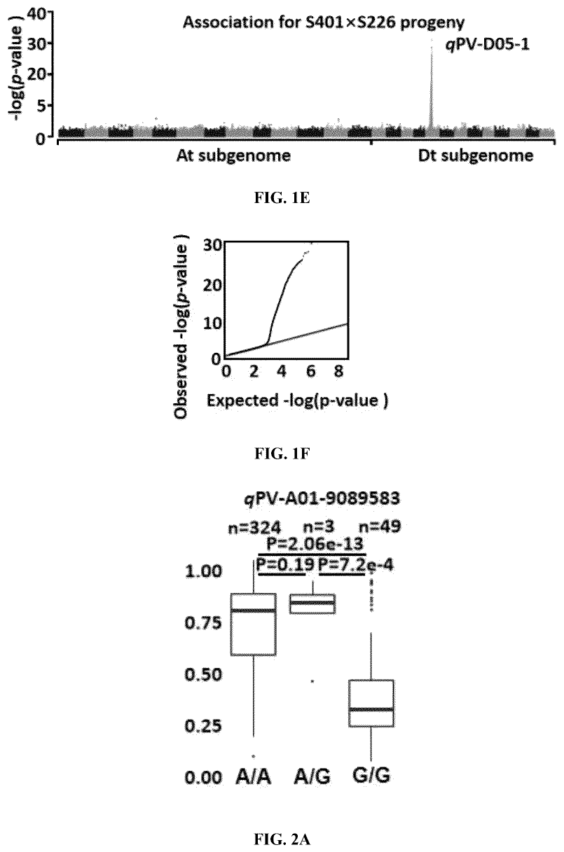

In the GWAS, a kinship matrix was constructed using the natural variation map to control false association signals generated during the association analysis.
3. A QTL interval was determined based on the position of the most significant SNP associated with the anther heat tolerance phenotype and linkage disequilibrium information (Table 1).
4. Two extremely high-temperature sensitive and two extremely heat-tolerant materials were selected from 376 upland cotton materials. Two pairs of F2 segregation generations were constructed using the above four materials for association analysis to verify the authenticity of QTL. The results are shown in FIGS. 1A-F (FIG. 1A, FIG. 1C and FIG. 1E were Manhattan plots for identifying four heat tolerance-related QTLs; FIG. 1B, FIG. 1D and FIG. 1F were Q-Q plots corresponding to the association analysis results) and Table 1. NOTE: the extremely high-temperature sensitive materials were Jimian 6/S151 and Ekangmian 8/S401, while the extremely heat-tolerant materials were Xinluzao 19/S9 and Yuanmian 5/S226.

TABLE 1
Genomic information and favorable alleles of four QTLs for heat tolerance

| | | Most | | | Most | |
|---|---|---|---|---|---|---|
| | | significantly- | | | significantly- | Heat- |
| | | associated | | | associated | tolerant |
| QTL name | Chromosome | SNP | QTL start | QTL end | SNP alleles | alleles |
| qPV-A01-1 | A01 | 9089583 | 8809583 | 9369583 | A/G | A/A |
| qPV-D01-1 | D01 | 8385035 | 8105035 | 8665035 | T/G | T/T |
| qPV-D05-1 | D05 | 23825179 | 23545179 | 24105179 | G/A | G/G |
| qPV-D12-1 | D12 | 42661640 | 42381640 | 42941640 | A/T | A/A |

As shown in FIGS. 1A-IF, association analysis based on natural populations of upland cotton identified three QTLs in the genome that were significantly associated with the anther heat tolerance phenotype, named qPV-A01-1, qPV-D01-1, and qPV-D12-1. Based on association analysis of two F2 segregating generations, two QTLs were identified in the genome, named qPV-A01-1 and qPV-D05-1. Through the analysis of three groups of materials, a total of 4 QTLs related to the heat tolerance phenotype of anthers were identified in the upland cotton genome.

As shown in Table 1, there were 4 QTLs significantly associated with the anther heat tolerance phenotype in the upland cotton genome, located on chromosomes A01, D01, D05, and D12. According to the QTL naming rules and linkage disequilibrium in the upland cotton genome, the above four QTL intervals were determined and named qPV-A01-1, qPV-D01-1, qPV-D05-1, and qPV-D12-1, respectively.

### Example 2

Analysis of the Effects and Utilization of Four QTLs Associated with Heat Tolerance in Natural Populations of Upland Cotton

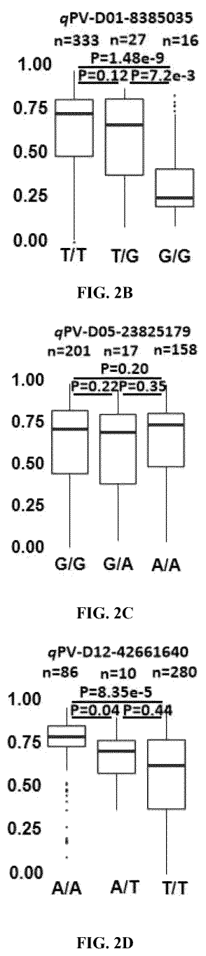

Based on the QTL interval determined in Example 1, the SNP variation most associated with the heat tolerance phenotype in upland cotton anther in qPV-A01-1, qPV-D01-1, qPV-D05-1, and qPV-D12-1 was used as the QTL genotype and displayed in the format of ‘QTL-SNP physical position-genotype’. The 376 upland cotton materials with corresponding QTL genotypes were grouped and statistically tested. The results are shown in FIGS. 2A-D (FIG. 2A represented the genotype effect analysis of qPV-A01-9089583-A/G, FIG. 2B represented the genotype effect analysis of qPV-D01-8385035-T/G, FIG. 2C represented the genotype effect analysis of qPV-D05-23825179-G/A, and FIG. 2D represented the genotype effect analysis of qPV-D12-42661640-A/T).

As shown in FIGS. 2A-2D, the genotypes of the above 4 anther heat tolerance QTLs were qPV-A01-9089583-A/G, qPV-D01-8385035-T/G, qPV-D05-23825179-G/A, and qPV-D12-42661640-A/T. After classifying different materials by genotype, it was found that the qPV-A01-9089583-A, qPV-D01-8385035-T, qPV-D05-23825179-G, and qPV-D12-42661640-T had a higher allele frequency in natural populations, and the corresponding homozygous genotype materials were 324 parts, 333 parts, 201 parts, and 280 parts, respectively. The qPV-A01-9089583-A, qPV-D01-8385035-T, qPV-D05-23825179-G, and qPV-D12-42661640-A were heat-tolerant genotypes, and the mean values of the heat tolerance phenotypes of the anthers of the corresponding materials were relatively high, and there were statistically significant differences except for qPV-D05-23825179-G.

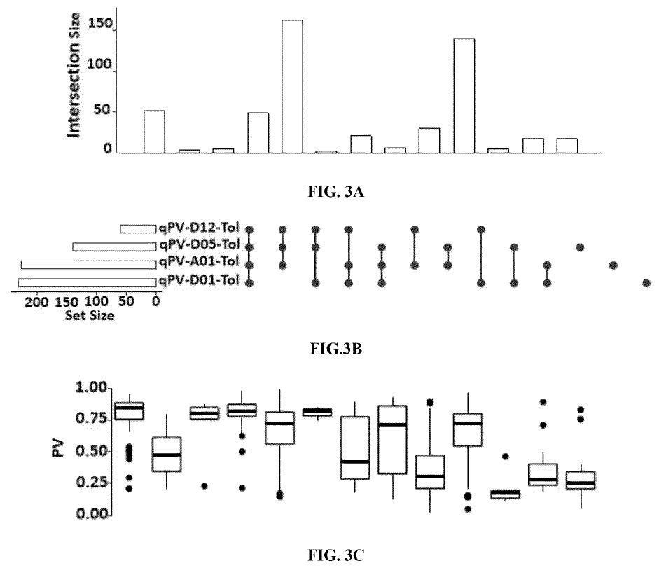

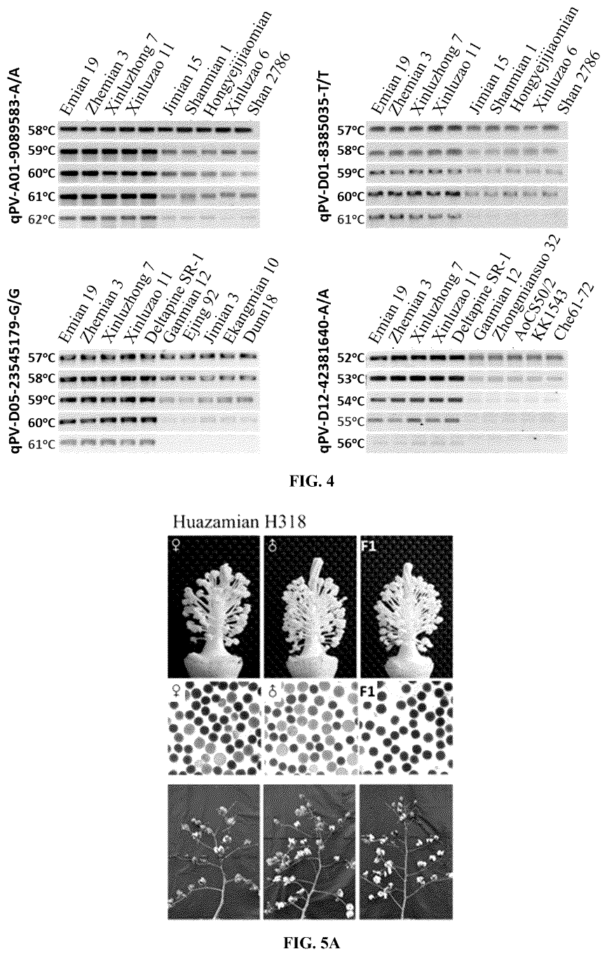

In addition, the combination of heat-tolerant QTL genotypes and the corresponding anther heat-tolerant phenotypes in 376 upland cotton samples were analyzed. The results are shown in FIGS. 3A-3C (the bar plot in FIG. 3A showed the number of materials corresponding to different QTL genotype combinations; the dot-line plot and bar plot in FIG. 3B showed the number of different heat-tolerant QTL genotypes in natural populations; the box-and-whisker plot in FIG. 3C showed the heat-tolerant phenotype values of upland cotton under different QTL genotype combinations).

As shown in FIGS. 3A-3C, the materials that simultaneously integrated four heat tolerance QTLs had desirable pollen vigor under high-temperature stress, but there were relatively small number of such materials, only more than 50. At the same time, there was no material using the heat-tolerant genotype of qPV-D12-42661640-A alone in natural populations.

### Example 3

Development of Molecular Markers for Four QTLs Associated with Heat Tolerance

In order to determine the authenticity of the heat tolerance QTLs and develop molecular markers, it was attempted to develop the four SNP variations of qPV-A01-9089583-A, qPV-D01-8385035-T, qPV-D05-23825179-G, and qPV-D12-42661640-A into annealing-sensitive KASP markers. According to the variation map in Example 1 and the 376 materials and corresponding genotype information in Example 2, the corresponding primers were designed. The principles and steps were as follows:
1. 200 bp each of genomic sequences upstream and downstream of the physical position of the corresponding SNP variations were downloaded (where specific nucleotides were set forth in SEQ ID NO: 1, SEQ ID NO: 3, SEQ ID NO: 5, and SEQ ID NO: 7, respectively). The primers at the target SNP position were designed, the target SNP was designed at the 3′-end of the target primer, and an annealing temperature was set a 52° C. to 54° C. The specific primer information was as follows:
an upstream primer had the nucleotide sequence set forth in SEQ ID NO: 9 and a downstream primer had the nucleotide sequence set forth in SEQ ID NO: 10 for amplifying the qPV-A01-9089583-A/G;
an upstream primer had the nucleotide sequence set forth in SEQ ID NO: 11 and a downstream primer had the nucleotide sequence set forth in SEQ ID NO: 12 for amplifying the qPV-D01-8385035-T/G;
an upstream primer had the nucleotide sequence set forth in SEQ ID NO: 13 and a downstream primer had the nucleotide sequence set forth in SEQ ID NO: 14 for amplifying the qPV-D05-23825179-G/A;
an upstream primer had the nucleotide sequence set forth in SEQ ID NO: 15 and a downstream primer had the nucleotide sequence set forth in SEQ ID NO: 16 for amplifying the qPV-D12-42661640-A/T (NOTE: the base A at the 3′-end of each upstream primer could be used to detect the target SNP; a design principle of the downstream primer was relatively loose and only needed to meet the annealing temperature of 52° C. to 54° C. to react and the product fragment was 150 bp to 200 bp).
2. After completing the primer design, according to the variation map information in Example 1, 5 materials containing the corresponding SNP variation were selected. The specific information of the upland cotton variety materials was shown in Table 2:

TABLE 2
Material information of upland cotton

| Material name | Common name | Source |
|---|---|---|
| Emian19 | Emian 19 | Independently bred in China |
| Zhemian3 | Zhemian 3 | Independently bred in China |
| Xinluzhong7 | Xinluzhong 7 | Independently bred in China |
| Xinluzao11 | Xinluzao 11 | Independently bred in China |
| DeltapineSR-1 | Deltapine | Introduced from American cotton |
| | cotton SR-1 | |
| Jimian15 | Jimian 15 | Independently bred in China |
| ShaanMian1 | Shanmian 1 | Independently bred in China |
| Hongyejijiaomian | Hongyejijiaomian | Independently bred in China |
| Xinluzao6 | Xinluzao 6 | Independently bred in China |
| Shaan2786 | Shan 2786 | Independently bred in China |
| Ganmian12 | Ganmian 12 | Independently bred in China |
| Ejing92 | Ejing 92 | Independently bred in China |
| Jimian3 | Jimian 3 | Independently bred in China |
| Ekangmian10 | Ekangmian 10 | Independently bred in China |
| Dunn18 | Dunn18 | Introduced from American cotton |
| Zhongmiansuo32 | Zhongmiansuo 32 | Independently bred in China |
| AoCS50/2 | Ao CS50/2 | Introduced from Australian cotton |
| KK1543 | KK1543 | Introduced from Soviet cotton |
| Che61-72 | Che 61-72 | Independently bred in China |

NOTE: in Table 2, five materials integrating four heat-tolerant genotypes (qPV-A01-9089583-G, qPV-D01-8385035-G, qPV-D05-23825179-A, and qPV-D12-42661640-T) were: Emian19, Zhemian3, Xinluzhong7, Xinluzao11, and DeltapineSR-1; five materials of qPV-A01-9089583-G genotype: Jimian15, ShaanMian1, Hongyejijiaomian, Xinluzao6, and Shaan2786; five materials of qPV-D01-8385035-G genotype: Jimian15, ShaanMian1, Hongyejijiaomian, Xinluzao6, and Shaan2786; five materials of qPV-D05-23825179-A genotype: Ganmian12, Ejing92, Jimian3, Ekangmian10, and Dunn18; five materials of qPV-D12-42661640-T genotype: Ganmian12, Zhongmiansuo32, AoCS50/2, KK1543, and Che61-72.

3. The genomic DNA of each material in Table 2 was extracted by a CTAB method. The fresh leaves were placed in a 2 mL centrifuge tube, added with clean steel beads and 200 μL extraction buffer (the extraction buffer was water-based, containing 0.35 M glucose, 0.1 M Tris-HCl, 5 mM Na₂EDTA, and 2% PVP K-30 (20 g dissolved in 1 L buffer), and 0.1% DIECA (1 g dissolved in 1 L buffer), pH=7.5), ground on a grinder (Shanghai Jingxin #Tissuelyser-192) for 60 s at a frequency of 60 Hz; after grinding, 800 μL lysis buffer was added (the lysis buffer was water-based, containing 0.1 M Tris-HCl, 1.4 M NaCl, 0.02 M Na₂EDTA, 2% CTAB, 2% PVP K-30, and 0.1% DIECA, pH=8.0) to the centrifuge tube; the above centrifuge tube was placed in a 65° C. water bath for 30 min, 800 μL of chloroform 24:1 was added (i.e., a mixture of chloroform and isoamyl alcohol with a volume ratio of 24:1), gently inverted, and then extracted for 20 min; after centrifugation at 12,000 rpm for 8-10 min, the supernatant was transferred and mixed with an equal volume of −20° C. pre-cooled isopropanol; after mixing, flocculent DNA precipitates appeared; the DNA was washed twice with 75% volume concentration of ethanol, blow-dried in a clean bench, and the DNA was dissolved with ddH₂O to obtain genomic DNA.

The extracted genomic DNA was amplified by PCR using the primers in step 1, a PCR amplification system was configured according to the formula in Table 3, and PCR amplification was conducted according to the program in Table 4.

TABLE 3
PCR reaction system

| Component | Volume | |
|---|---|---|
| Genomic DNA | 1 μL (75-100 ng loading volume) | |
| 10× Buffer | 2 | μL |
| Upstream primer (10 μM) | 0.5 | μL |
| Upstream primer (10 μM) | 0.5 | μL |
| dNTP mix | 0.3 | μL |
| Taq enzyme | 0.2 | μL |
| ddH₂O | 15.5 | μL |
| Total volume | 20 | μL. |

TABLE 4
PCR reaction procedure

| | | |
|---|---|---|
| Step | Temperature | Time |

| Initial denaturation | 95° | C. | 5 min each time |
|---|---|---|---|
| Denaturation | 95° | C. | 30 s |
| Annealing | 53-62° | C. | 30 s |
| Extension | 72° | C. | 30 s |
| Cycle | 35 cycles from denaturation to extension | | |
| Final extension | 72° | C. | 30 s |

4. The annealing temperature ranging from 53° C. to 62° C. was set in the PCR amplification program to evaluate the optimal annealing temperature for detecting molecular markers. For example, for the qPV-A01-9089583-A marker, at a certain annealing temperature, the detection primer could still anneal and extend normally with the reverse sequence of the target base A, and finally produce a positive result. In addition, the remaining bases could not anneal normally, and the Taq enzyme could not start the extension, which reduced the PCR amplification efficiency or fails, and produced a negative result. The specific results were shown in FIG. 4.

As shown in FIG. 4, qPV-A01-9089583-A, qPV-D01-8385035-T, qPV-D05-23825179-G, and qPV-D12-42661640-A were developed as KASP markers, and their optimal annealing temperatures were 62° C., 61° C., 61° C. and 55° C., respectively. The qA01-Tolerant, qD01-Tolerant, qD05-Tolerant, and qD12-Tolerant were used to represent the dominant genotypes of qPV-D01-8385035-TT, qPV-D05-23825179-GG, and qPV-A01-9089583-AA, qPV-D12-42661640-AA, respectively.

### Example 4

Molecular Marker Verification of QTLs for Heat Tolerance and Breeding of Cotton Varieties with Heat Tolerance

The PCR reaction system, reaction procedure, primer sequence, and annealing temperature in Example 3 were used to verify commercial cotton varieties. The specific process was as follows:

The heat-tolerant hybrid cotton variety ‘Huazamian H318’ that had been approved by the laboratory earlier (Huazamian H318 was approved in 2009, with the approval number of Guo Shen Mian 2009018, and the hybrid variety was made public in DOI: 10.1186/s12864-021-07580-8) and the corresponding hybrid parent materials of the hybrid cotton variety (female parent ‘B0011’ and male parent ‘4-5’) were selected for marker verification; the phenotypes of the hybrid F1 and parents of ‘Huazamian H318’ in the field were statistically analyzed, and the results are shown in FIG. 5A. In FIG. 5A, the first row showed the anther morphology of the female parent (mark), male parent (mark) and F1 hybrid (F1 mark) of H318, a Chinese cotton plant, after high-temperature stress; the second row showed the pollen microscopic staining of the female parent, male parent and F1 hybrid of H318, a Chinese cotton plant; the third row showed the plant type characteristics of the female parent, male parent and F1 hybrid of H318, a Chinese cotton plant, at the harvest stage.

As shown in FIG. 5A, ‘Huazamian H318’ had an obvious heat tolerance phenotype in the field, which was significantly different from the heat tolerance performance of its parents, and served as a desirable object for marker verification.

PCR amplification was conducted using the 4 KASP markers developed in Example 3, namely qPV-A01-9089583-A, qPV-D01-8385035-T, qPV-D05-23825179-G, and qPV-D12-42661640-A, at optimal annealing temperatures of 62° C., 61° C., 61° C., and 55° C., respectively. The results are shown in FIG. 5B.

As shown in FIG. 5B, the female parent ‘B0011’ line of ‘Huazamian H318’ contained four QTLs for heat tolerance, and the male parent ‘4-5’ line contained two QTLs for heat tolerance. The genotypes of the four heat tolerance QTLs were different in the maternal and paternal genomes of ‘Huazamian H318’, but the hybrid F1 ‘Huazamian H318’ aggregated the four heat tolerance QTLs, which were consistent with the heat tolerance phenotype in the field (FIG. 5A). It was seen that the heat tolerance QTL genotypes of different materials could be identified by PCR, indicating that the four heat tolerance QTLs identified in the present disclosure did exist in upland cotton materials and the KASP marker was truly usable.

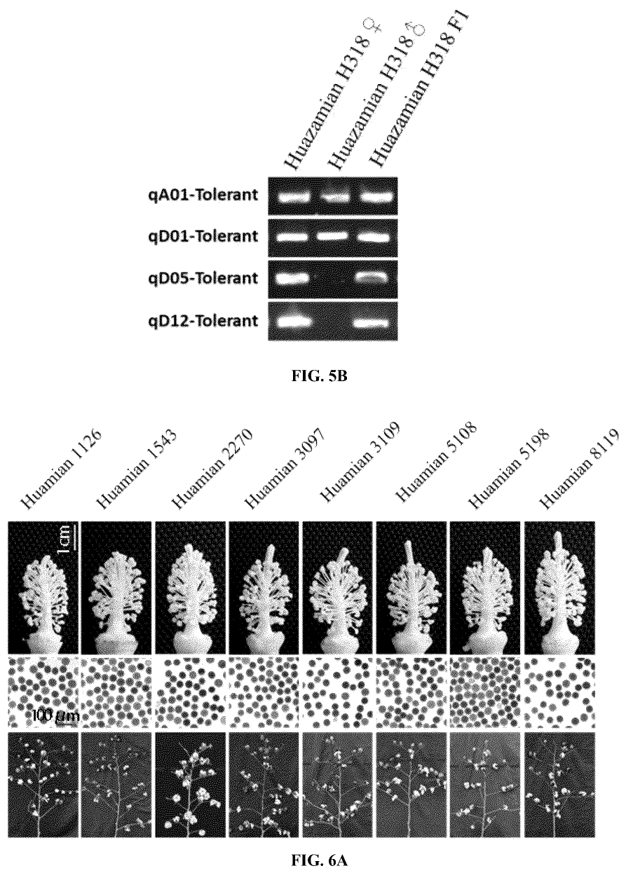

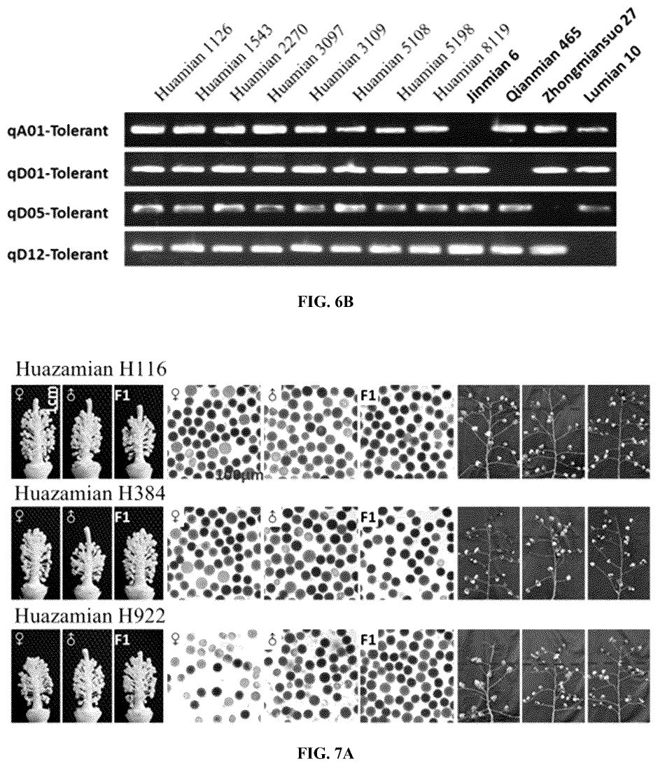

Based on the genetic characteristics of the heat-tolerant QTL, conventional heat-tolerant high-quality cotton varieties were selected in combination with comprehensive quality and yield phenotypes: ‘Huamian 2270’, ‘Huamian 5108’, ‘Huamian 1126’, ‘Huamian 1543’, ‘Huamian 3097’, ‘Huamian 5198’, ‘Huamian 8119’, and ‘Huamian 3109’ were used as samples to be tested, and Jinmian6, Qianmian465, Zhongmiansuo27 and Lumian10 were used as negative controls for qA01-Tolerant, qD01-Tolerant, qD05-Tolerant, and qD12-Tolerant markers, respectively, to allow PCR verification (the PCR reaction system was the same as Table 3, and the PCR reaction procedure was the same as Table 4). The results are shown in FIGS. 6A-6B (in FIG. 6A, the first row showed the anther pictures of 8 conventional species in the Huamian series after high-temperature stress, the above materials could still crack and release pollen normally after high-temperature stress; the second row showed the microscopic pictures of pollen of 8 conventional species in the Huamian series after high-temperature stress; corresponding to the anther phenotype, the above materials could all produce high-vitality fertile pollen normally after high-temperature stress; the third row showed the typical single plant performance of 8 conventional species in the Huamian series; after the above materials encountered high-temperature stress in the field, the boll-bearing ability was almost not affected, and the fruit branches that were most severely affected by high-temperature stress (upper part of the black line) could still produce bolls normally. As shown in FIG. 6B, the approved conventional heat-tolerant varieties Huamian 1126, 1543, 2270, 3097, 3109, 5108, 5198, and 8119 all contained 4 heat-tolerant QTLs, and also had obvious heat-tolerant characteristics in the performance of individual plants in the field (shown in the third row of pictures in FIG. 6A), corresponding to their genotypes.

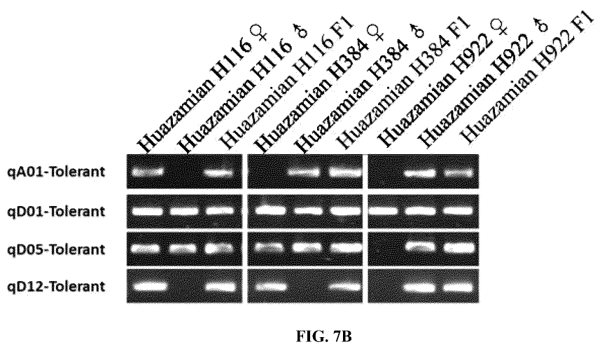

Utilizing hybrid vigor was one of the important goals of cotton variety breeding. The laboratory combined molecular markers and superior parents to breed the hybrids of ‘Huazamian H116’, ‘Huazamian H834’, and ‘Huazamian H922’. The corresponding hybrid F1 was observed for field tolerance to high temperature. The results are shown in FIG. 7A. Jinmian6, Qianmian465, Zhongmiansuo27, and Lumian10 were used as negative controls for qA01-Tolerant, qD01-Tolerant, qD05-Tolerant, and qD12-Tolerant markers, respectively, to allow PCR verification (the PCR reaction system was the same as Table 3, and the PCR reaction procedure was the same as Table 4). The results are shown in FIG. 7B.

As shown in FIG. 7A, the hybrid F1 of ‘Huazamian H116’, ‘Huazamian H834’, and ‘Huazamian H922’ showed obvious tolerance to high temperature in the field; in FIG. 7A, the first row showed the performance of anthers and pollen per plant of ‘Huazamian H116’ and its two parents (the female parent was indicated by the mark, the male parent was indicated by the mark, and the hybrid was indicated by the F1 mark) after high-temperature stress; the second row showed the performance of anthers and pollen per plant of ‘Huazamian H834’ and its two parents after high-temperature stress; the third row showed the performance of anthers and pollen per plant of ‘Huazamian H922’ and its two parents after high-temperature stress. The black lines in FIG. 7A indicated the parts of the fruit branches that were most severely subjected to high-temperature stress in the field.

As shown in FIG. 7B, the female parent ‘H82140’ line of ‘Huazamian H116’ contained 4 QTLs for heat tolerance, and the male parent ‘H92047’ line contained two QTLs for heat tolerance; the female parent ‘Jingyin511’ line of ‘Huazamian H834’ contained 3 QTLs for heat tolerance, and the male parent ‘H92072’ line contained 3 QTLs for heat tolerance; the female parent ‘361-9’ line of ‘Huazamian H922’ contained 1 QTL for heat tolerance, and the male parent ‘4007-17’ line contained four QTLs for heat tolerance. Although not all parents contained all the QTLs for heat tolerance, through hybridization, the hybrid F1 had all the QTLs for heat tolerance, and had both parental heterosis and heat tolerance phenotype.

In summary, the technical solutions provided by the present disclosure can accurately identify and screen the heat tolerance trait of upland cotton and then achieve the breeding of an ideal cotton plant type.

Although the present disclosure is described in detail in conjunction with the foregoing examples, they are only a part of, not all of, the examples of the present disclosure. Other examples can be obtained based on these examples without creative efforts, and all of these examples shall fall within the protection scope of the present disclosure.

What is claimed is:

1. (canceled)

2. (canceled)

3. (canceled)

4. A primer set for amplifying a molecular marker, wherein the primer set is one or more selected from the group consisting of a primer set for amplifying the qPV-A01-9089583-A/G, a primer set for amplifying the qPV-D01-8385035-T/G, a primer set for amplifying the qPV-D05-23825179-G/A, and a primer set for amplifying the qPV-D12-42661640-A/T;
an upstream primer has the nucleotide sequence shown in SEQ ID NO: 9 and a downstream primer has the nucleotide sequence shown in SEQ ID NO: 10 in the primer set for amplifying the qPV-A01-9089583-A/G;
an upstream primer has the nucleotide sequence shown in SEQ ID NO: 11 and a downstream primer has the nucleotide sequence shown in SEQ ID NO: 12 in the primer set for amplifying the qPV-D01-8385035-T/G;
an upstream primer has the nucleotide sequence shown in SEQ ID NO: 13 and a downstream primer has the nucleotide sequence shown in SEQ ID NO: 14 in the primer set for amplifying the qPV-D05-23825179-G/A; and
an upstream primer has the nucleotide sequence shown in SEQ ID NO: 15 and a downstream primer has the nucleotide sequence shown in SEQ ID NO: 16 in the primer set for amplifying the qPV-D12-42661640-A/T;
wherein the molecular marker is one or more selected from the group consisting of qPV-A01-9089583-A/G, qPV-D01-8385035-T/G, qPV-D05-23825179-G/A, and qPV-D12-42661640-A/T;
the qPV-A01-9089583-A/G is located at a base 9,089,583 of the chromosome A01 in the genome of the upland cotton, and the base 9,089,583 is A or G;
the qPV-D01-8385035-T/G is located at a base 8,385,035 of the chromosome D01 in the genome of the upland cotton, and the base 8,385,035 is T or G;
the qPV-D05-23825179-G/A is located at a base 23,825,179 of the chromosome D05 in the genome of the upland cotton, and the base 23,825,179 is G or A; and
the qPV-D12-42661640-A/T is located at a base 42,661,640 of the chromosome D12 in the genome of the upland cotton, and the base 42,661,640 is A or T;
the molecular marker tightly linked to a quantitative trat locus (QTL) associated with heat tolerance in an upland cotton, wherein the QTL is one or more selected from the group consisting of qPV-A01-1, qPV-D01-1, qPV-D05-1, and qPV-D12-1;
the qPV-A01-1 is located at 8,809,583 bp to 9,369,583 bp of a chromosome A01 in a genome of the upland cotton;
the qPV-D01-1 is located at 8,105,035 and 8,665,035 bp of a chromosome D01 in the genome of the upland cotton;
the qPV-D05-1 is located at 23,545,179 bp to 24,105,179 bp of a chromosome D05 in the genome of the upland cotton;
the qPV-D12-1 is located at 42,381,640 bp to 42,941,640 bp of a chromosome D12 in the genome of the upland cotton; and
the genome of the upland cotton has a version of TM-1_HZAU.v1.1.

5. A detection kit, comprising the primer set according to claim 4.

6. The detection kit according to claim 5, further comprising a PCR amplification reagent.

7. The detection kit according to claim 6, wherein the PCR amplification reagent comprises a DNA polymerase, dNTPs, and Mg²⁺.

8. (canceled)

9. (canceled)

10. A method for identifying a heat-tolerant upland cotton, comprising the following steps:
subjecting a genomic DNA of an upland cotton to be tested to PCR amplification using the primer set according to claim 4 to obtain a PCR amplification product; detecting and analyzing a genotype of the PCR amplification product;
if the molecular marker qPV-A01-9089583-A/G has a heat-tolerant homozygous genotype AA, the molecular marker qPV-D01-8385035-T/G has a heat-tolerant homozygous genotype TT, the molecular marker qPV-D05-23825179-G/A has a heat-tolerant homozygous genotype GG, and the molecular marker qPV-D12-42661640-A/T has a heat-tolerant homozygous genotype AA in the PCR amplification product, determining that the upland cotton to be tested is the heat-tolerant upland cotton; and
if at least one of the molecular markers qPV-A01-9089583-A/G, qPV-D01-8385035-T/G, qPV-D05-23825179-G/A, and qPV-D12-42661640-A/T has a non-heat-tolerant homozygous genotype in the PCR amplification product, determining that the upland cotton to be tested is a non-heat-tolerant upland cotton.

11. The method according to claim 10, wherein a reaction system of the PCR amplification has a volume of 20 μL and comprises: 2 μL of a 10× Buffer, 1 μL of a 75 ng/μL to 100 ng/μL genomic DNA template, 0.5 μL of a 10 μM upstream primer, 0.5 μL of a 10 μM downstream primer, 0.3 μL of a dNTP mix, 0.2 μL of a Taq enzyme, and supplementing to 20 μL with double distilled H₂O.

12. The method according to claim 10, wherein a reaction procedure of the PCR amplification comprises: initial denaturation at 95° C. for 5 min; 35 cycles of denaturation at 95° C. for 30 s, annealing at 53° C. to 62° C. for 30 s, and extension at 72° C. for 30 s; and extension at 72° C. for 30 s.

13. The method according to claim 12, wherein the qPV-A01-9089583-A/G is amplified at an annealing temperature of 62° C.; the qPV-D01-8385035-T/G is amplified at an annealing temperature of 61° C.; the qPV-D05-23825179-G/A is amplified at an annealing temperature of 61° C.; and the qPV-D12-42661640-A/T is amplified at an annealing temperature of 55° C.

14. The primer set according to claim 4, wherein the nucleotide sequence having the qPV-A01-9089583-A/G is set forth in SEQ ID NO: 1 or SEQ ID NO: 2;
the nucleotide sequence having the qPV-D01-8385035-T/G is set forth in SEQ ID NO: 3 or SEQ ID NO: 4;
the nucleotide sequence having the qPV-D05-23825179-G/A is set forth in SEQ ID NO: 5 or SEQ ID NO: 6; and
the nucleotide sequence having the qPV-D12-42661640-A/T is set forth in SEQ ID NO: 7 or SEQ ID NO: 8.

15. The method according to claim 12, wherein a reaction system of the PCR amplification has a volume of 20 μL and comprises: 2 μL of a 10× Buffer, 1 μL of a 75 ng/μL to 100 ng/μL genomic DNA template, 0.5 μL of a 10 μM upstream primer, 0.5 μL of a 10 μM downstream primer, 0.3 μL of a dNTP mix, 0.2 μL of a Taq enzyme, and supplementing to 20 μL with double distilled H₂O.

16. The method of claim 10, wherein the heat-tolerant upland cotton is tolerant to a temperature of greater than or equal to 35° C.
